(19) United States
(12) Patent Application Publication
(10) Pub. No.: US 2021/0389330 A1
(43) Pub. Date: Dec. 16, 2021

(54) NON-INVASIVE APPROACH TO EARLY DIAGNOSIS AND PROGNOSIS OF ACUTE SEVERE PANCREATITIS BASED ON HEAT SHOCK PROTEINS MECHANISM OF ACTION

(71) Applicant: Vilnius University, Vilnius (LT)

(72) Inventors: Kestutis Strupas, Vilnius (LT); Aiste Gulla, Vilnius (LT)

(21) Appl. No.: 17/291,625

(22) PCT Filed: Nov. 7, 2019
(86) PCT No.: PCT/IB2019/059573

§ 371 Date: May 6, 2021

(30) Foreign Application Priority Data
Nov. 7, 2018 (LT) LT2018 548

(51) Int. Cl. G01N 33/68

(52) U.S. Cl. CPC G01N 33/6893; G01N 2800/067

(57) ABSTRACT

This invention describes a method for early diagnostics of severe pancreatitis using cell membrane damage detection mechanisms. The method involves a non-invasive testing of human urine or other body fluids samples of patients that are suspected to be affected by acute pancreatitis. The invention is based on heat shock protein HSP-70, 90, 60, and 27 role of action in disease progression.

### FIELD OF INVENTION

Method for early diagnostics of severe pancreatitis using cell membrane damage detection mechanisms.

### BACKGROUND OF INVENTION

Acute pancreatitis (AP) is one of the most common diseases in gastroenterology and about 2% of all hospitalized patients are diagnosed with the disease (Fagenholz, Castillo, et al. 2007). The incidence of AP per 100,000 population ranges from 10 to 46 cases per year. However, neither the etiology nor the pathophysiology of the disease are fully understood and thus treatment options targeting a specific underlying cause remain elusive (Lerch and Gorelick 2013; Niederau, Ferrell, and Grendell 1985). This has sparked the interest into studying the initial triggering events of AP in order to develop novel treatments used in clinical practise.

Acute pancreatitis is mild in most cases and without serious complications. Nevertheless, it is associated with morbidity and mortality in up to 20% of patients (Lerch and Gorelick 2013; Bhatia et al. 2005). While AP has been intensively investigated for decades, yet the pathophysiology of the pancreatitis process is not fully understood and no specific or effective treatment has been developed (Lerch and Gorelick 2013; Sah and Saluja 2011).

While initial diagnosis is made by typical clinical symptoms, blood tests findings (serum pancreatic amylase or lipase is three times above the normal range) and radiological findings (abdominal ultrasound); thus the disease and its progress are confirmed by computer tomography, which is considered the gold standard and is performed 3-7 days after the symptoms start in late stages of acute pancreatitis. Later stage of the disease cause not only complex complications but also fatal outcomes. Meanwhile, variety of different internationally approved (i.e. Ranson criteria, modified Atlanta classification) severity scales are also used in clinical practice, usually performed 48 hours since the admission to the hospital. Currently, there are no non-invasive tests available in clinical settings used for AP early diagnosis or prognosis.

Heat shock proteins have been known for many years. Molecular chaperones are protein machines that are responsible for the correct folding, stabilization, and function of other proteins in the cell. Exposure of cells to environmental stress, including heat shock, alcohols, heavy metals or oxidative stress, results in the cellular accumulation of these chaperones, commonly known as heat shock proteins (Hsp's). Heat-shock protein 90 (Hsp90) constitutes about 1-2% of total cellular proteins and is usually present in the cell as a dimer. It is a molecular chaperone responsible for ATP-depended folding, stability and function of many “client” proteins that are involved in the development and progression of cancer. These client proteins include ErbB2, c-Raf, Cdk4, mutant p53, hTERT, Hifl-α, and the estrogen/androgen receptors. Inhibition of Hsp90 causes the simultaneous, combinatorial destabilization and degradation of the oncogenic client proteins, leading in turn to a multiprolonged attack on all of the hallmark traits of cancer, including unrestricted proliferation and survival, invasion, metastasis and angiogenesis. It is generally thought that cancer cells are more susceptible to Hsp90 inhibition than are the corresponding normal cells (Workman 2004). On the other hand, therapeutic selectivity of Hsp90 inhibitors is the stressed condition of cancer cells, due both to oncogenic mutations and deregulated signalling and also to environmental factors such as hypoxia, acidosis and nutrient deprivation. Moreover, it has been reported that the Hsp90 found in malignant cells exists predominantly in a superchaperone complex that binds Hsp90 inhibitors much more effectively than the uncomplexed form that is mainly present in healthy cells (Dymock et al. 2004).

According to Rakonczay et al. caerulein pancreatitis has been reported to increase (Weber et al. 1995; Ethridge et al. 2000; Bhagat et al. 2002) or decrease (Strowski et al. 1997, 1997; Z. J. Rakonczay, Mandi, et al. 2002) the synthesis of pancreatic HSP60 and HSP72. Ethridge et al. (2000) found that the pancreatic HSF-1 binding activity was increased during caerulein induced acute pancreatitis, with concomitant increases in HSP70 and HSP27, while the levels of HSP60 and HSP90 were not affected. Similarly, Weber et al. (1995) demonstrated that caerulein induced pancreatic HSP70 mRNA and protein expression. However, secretagogue treatment did not alter the level of ubiquitin mRNA. Using antisense oligonucleotides, Bhagat et al. (2002) showed that the caerulein-induced HSP72 expression acts to limit the severity of the disease in rats. Strowski et al. (1997) observed that caerulein time and dose dependency increases mRNA but paradoxically reduces protein levels of rat pancreatic HSP60 and HSC70. In accordance with their study, we have shown that repeated injections of supramaximal doses of cholecystokinin-octapeptide (CCK) for 5 days reduce the pancreatic HSP60 and HSP72 levels (Rakonczay et al., 2002d). The administration of nontoxic BRX-220 upregulated the synthesis of pancreatic HSP60 and HSP72 during the course of the disease (Z. J. Rakonczay, Ivanyi, et al. 2002).

Furthermore, the expression of pancreatic HSP32 mRNA is rapidly upregulated, while the protein level was only slightly increased during caerulein-induced acute pancreatitis in mice (Fu et al. 1997). Researchers have established that HSP32 was expressed at very low basal levels in the pancreas in vivo and in acinar cells in vitro (Sato et al. 1997). Caerulein-induced acute pancreatitis was associated with a significant increase in the expression of pancreatic HSP32 12-24 h after the first signs of interstitial edema in the pancreas of rats. Interestingly, the addition of caerulein to the culture medium of AR42J acinar cells had no effect on the HSP32 levels.

TABLE 3
Effects of acute pancreatitis, on pancreatic HSP levels

| Acute | | | |
|---|---|---|---|
| pancreatitis | Species | HSP levels | Reference |
| Cerulien | Mouse | ↑HSP27, HSP72 → HSP60, HSP90 | Ethridge et al., 2000 |
| Cerulien | Mouse | ↑HSP32 | Fu et al., 1997 |
| Cerulien | Rat | ↑HSP70 → ubiquitin | Weber et al., 1995 |
| Cerulien | Rat | ↑HSP70 | Bhagat et al., 2002 |
| Cerulien | Rat | ↓HSP60, HSC70 → HSP70 | Strowski et al., 1997; Weber et al., 2000 |
| CCK | Rat | ↓HSP60, HSP72 | Rakoneray et al., 2002d |
| Cerulien | Rat | ↑HSP32 | Sato et al., 1997 |
| DBTC | Rat | ↑HSP70, HSC70 | Weber et al., 2000 |
| Arg | Rat | ↑HSP27, HSP72, HSP30, HSP90 → GRP78 | Tashiro et al., 2002 |
| CDE | Mouse | →HSP70 | Grise et al., 2000 |
| CDE | Mouse | ↓HSP27, HSP70 | Delhaye et al., 2000 |

↑, increased;
↓, decreased;
→, no change;
CCK, cholecystokinin-octapeptide;
DBTC, dibutyltin dichloride;
CDE, choline-deficient ethiotine-supplemented diet;
Arg, arginine.

As recently published by Gini et al., one of the most conserved and well-studied family of heat shock proteins is HSP70 superfamily. According to the authors, these are ATP dependent chaperones that range in size from 66 to 78 kDa and are encoded by a set of 11 genes in humans. Interesting, the authors reported that the levels of these proteins are transcriptionally regulated by Heat Shock Factors (HSF) which consists of four members: HSF1, HSF2, HSF3, and HSF4, therefore they possess unique and overlapping functions and have tissue-specific patterns of expression (Gini et al. 2017).

As described in the previously mentioned references, multiple studies have evaluated the protective role of heat shock proteins in acute pancreatitis. The publication describes various studies have used various methods, including thermal stress, to overexpress HSP70. Wagner et al. found that when acini from rat pancreas were exposed to heat, markedly increased expression of the 72 kDa HSP-70 molecule was observed. Interestingly, the authors observed that hyperthermia induced HSP70 overexpression protected against organ damage in caerulein induced pancreatitis model. Similar results were observed even in models where HSP70 was induced via non-thermogenic methods. For example, over-expression of HSP70 by beta-adrenergic stimulation protected mice from caerulean induced pancreatitis. Similarly, administration of sodium arsenite induced overexpression of HSP70 provided protection caerulean as well as in arginine-induced model of acute pancreatitis (Bhagat et al. 2008).

As reported by Gini et al., these findings overwhelmingly suggest that HSP70 is protective against acinar cell injury in pancreatitis and that this protection is independent of the mode of induction of HSP70. Although, HSP70 was the major protein induced by heat stress as well as pharmacologic manipulation, these interventions induce multiple HSPs and it is possible that the protective effects observed with these interventions are due to events other than overexpression of HSP70. This was investigated by Bhagat et al. who showed that HSP70 overexpression is in fact responsible for the observed protective role of heat stress using in vitro culture of pancreatic lobules.

Multiple publications have shown that HSPs are differentially expressed in human diseases such as pancreatic adenocarcinoma, chronic pancreatitis and the normal pancreas. HSP90a mRNA was selectively overexpressed in pancreatic carcinoma (Gress et al. 1994; Ogata et al. 2000). HSP70 mRNA levels were increased in pancreatic adenocarcinoma and chronic pancreatitis (Gress et al. 1994).

Acute pancreatitis is an autodigestive process caused by pancreatic proteolytic enzymes extravasation, leading to contiguous tissue damage (Peery et al. 2012; Fagenholz, Ferna\acute{ }ndez-del Castillo, et al. 2007). Severe pancreatitis may induce a systemic inflammatory response syndrome and multiple organ damage (Fagenholz, Ferna\acute{ }ndez-del Castillo, et al. 2007). The inflammatory response involves both cellular and humoral factors, including cytokines, lipid mediators, and reactive oxygen species. The lung is often a target organ of inflammatory injury associated with severe acute pancreatitis. Acute lung injury is characterized by increased vascular permeability and neutrophil (polymorphonuclear leukocyte (PMN)) activation followed by migration (Fagenholz, Ferna\acute{ }ndez-del Castillo, et al. 2007). Activated neutrophils release enzymes, as for instance, matrix metalloproteinase (MMP) enzymes, to destroy bacteria and to remove damaged tissue. Matrix metalloproteinases act in processing matrix proteins, cytokines, growth factors, and adhesion molecules (Z. Rakonczay et al. 2003). Furthermore, the authors describe the role of Heat Shock Proteins during an inflammatory process which assumed a major interest because Heat Shock Proteins were shown to inhibit the expression of proinflammatory genes and to have a citoprotective effect on experimental models (Whitcomb 2004). More detailed information on the protective role of HSP 70 was described by Dudeja et al. (Dudeja, Vickers, and Saluja 2009).

The role of HSPs in protection against pancreatitis has been explored in various in vitro and in vivo models of pancreatitis. In accordance with the protective role of HSPs against injury, it has been shown that prior induction of HSP60, which is induced in rats by the stress of water immersion, protects against pancreatitis (Lee et al. 2000). In fact, all the markers of severity of pancreatitis—hyperamylasemia, pancreatic oedema and acinar cell necrosis—were reduced by this prior induction of HSP60 (Lee et al. 2000). The protective effect of HSPs in pancreatitis is not limited to HSP60, but is also observed with HSP70 induction. Since cellular injury in pancreatitis is mediated by both necrosis and apoptosis pathways and since HSP70 can protect cells against both necrotic and apoptotic programmed cell death, it is probable that HSP70 protects against both necrotic and apoptotic cell death during pancreatitis (Feng and Li 2010). Further development and investigation is needed.

It appears that this induction of HSP70 in the pancreas by the stress of pancreatitis is protective against pancreatitis, since prevention of this increase in HSP70 by administration of the antisense HSP70 in itself aggravates the severity of pancreatitis (Bhagat et al. 2002). The specific role of HSP70 in protection against pancreatitis is further proved by the use of heat shock factor-1 (HSF-1) knock-out mice and HSP70 transgenic mice (Dudeja, Vickers, and Saluja 2009). HSF-1 is the transcription factor for HSP70. Thus, HSF-1 knock-out animals lack the ability to express HSP70. Dudeja et al, 2009 similarly describes, HSP70 transgenic mice, which demonstrate markedly increased expression of HSP70, have reduced severity of pancreatitis.

Some progress has been made in elucidating how HSPs protect against pancreatitis (Feng and Li 2010). According to (Iovanna and Ismailov 2009) it has been shown that HSP70 induction prevents activation of trypsinogen to trypsin, a hallmark event of pancreatitis. Rakonczay et al. have shown that HSP70 induction does not protect against the pancreatitis induced by trypsin injection into the pancreas, again suggesting that HSP70 mediates protection against pancreatitis by influencing the events before trypsin activation. Also, evidence suggests that HSP70 influences the trypsinogen activation and the other downstream events during pancreatitis by preventing the co-localisation of lysosomal enzyme cathepsin B and the digestive enzyme zymogen (Bhagat et al. 2002). The events regulating co-localisation are less clear. However, evidence strongly suggests that cytosolic calcium is required for this process (Dudeja, Vickers, and Saluja 2009). Stimulation of pancreatic acini with a supramaximal dose of caerulein leads to marked elevation of cytosolic calcium followed by prolonged sustained elevation of cytosolic calcium maintained by calcium influx into the cell. According to Dudeja et al. 2009, some other studies in non-pancreatic acinar cells have also suggested that HSPs could influence calcium homeostasis in the cell. Thus, one of the mechanisms by which HSPs could influence co-localisation and the subsequent downstream events in pancreatitis is by attenuating cytosolic calcium. Given the importance of calcium in regulation of cellular processes including cell death and apoptosis, the finding that HSPs could influence calcium homeostasis is of much broader importance than just of significance to the pathophysiology of pancreatitis (Dudeja, Vickers, and Saluja 2009).

### SUMMARY OF THE INVENTION

We have investigated cell membrane wall damage mechanisms that cause systemic responses to acute severe pancreatitis course while creating non-invasive acute pancreatitis diagnostic and prognostic methods. For this approach we are applying tethered bilayers lipid membranes (McGillivray et al. 2007; Budvytyte et al. 2013; Ragaliauskas et al. 2017). In addition, identification of heat shock proteins-27, 32, 60, 70, 90 associated with severe acute pancreatitis mechanisms can be used as a biomarkers and as a diagnostic kit, while establishing non-invasive diagnostic approaches.

Our experiments were based on HSP-70 and HSP-90 (Cikotiene et al. 2009; Budvytyte et al. 2013; Ragaliauskas et al. 2017). Here we provide the summary of the existing levels of Heat shock protein-70 and Heat shock protein-90 measuring levels and assays reported in the literature. To the best of our knowledge, there is no HSP-70 or HSP-90 tested in acute pancreatitis based on non-invasive methodology. We're also investigating further different combinations of HSPs that include but are not limited to: HSP 27 and HSP 32, also HSP 32 and HSP70, HSP70 and HSP90, HSP60 and HSP70, HSP60 and HSP90, HSP27 and HSP90.

### DESCRIPTION OF DRAWINGS

FIG. 1. Interaction between HSP70 and HSP90 with tBLM. A—Phase angle plot of EIS spectra: black—the initial spectra of tBLM, green—10 min after HSP70 of 0.2 uM injected, red—after 24 hours after injection of HSP70. tBLM was composed from DOPC and Cholesterol (in ratio % 60:40). B.—Phase angle plot of EIS spectra: black—the initial spectra of tBLM, green—10 min after HSP90 of 2 uM injected, red—after 24 hours after injection of HSP90. tBLM was composed from DOPC and Cholesterol (in ratio % 75:25).

FIG. 2. SPR sensograms of the adsorptions of HSP70 and HSP90 proteins on lipid membranes (DOPC:cholesterol in ratios 60:40 and 75:25, respectively). HSP70 and HSP90 concentrations are x and y, respectively.

### DETAILED DESCRIPTION OF THE INVENTION

Our invention involves using the methodology shown in patent LT 6424 entitled “Method of preparing immobilized on the surface phospholipid bilayer membrane (TBLM)” and applying approved methods in acute pancreatitis early diagnosis settings.

In one embodiment of the invention a prognostic and diagnostic biomarker or set biomarkers (diagnostic kit) are assembled for detection of acute pancreatitis by measuring Heat Shock Proteins (HSP) as major players in the mechanism of the severity of acute pancreatitis. The samples for testing are taken patients' urine, sweat, or saliva and blood serum/plasma levels are used as controls.

In another embodiment, the invention can serve as a therapeutic target to determine early multiple organ dysfunction syndrome in the setting of acute pancreatitis.

### Materials and Methods

### HSP-70

Recombinant Protein HSP70 was purchased from Sigma Aldrich (Germany) with purity of ≥70%. Recombinant HSP90 full length was donated by prof. D. Matulis from VU Life Sciences Center, Institute of Biotechnology, HSP90 was cloned and purified.

Serum HSP70 was analysed using Human Heat Shock Protein 70 (HSP70), (Cat no: 20,959) purchased from Bio Medical Assay (BMASSAY, Beijing, China) following the manufacturer's instructions. Their levels were expressed as ng/ml. The detection and quantification limits were set at <0.05 ng/ml for HSP70.

Serum HSP70 protein levels were determined using commercially available ELISA kits (StressGen Biotechnologies Corp). The concentrations of HSP70 protein were determined by comparison with a standard curve according to manufacturer's direction. The standard curve has a range of 0.78 to 50 ng/mL, and the sensitivity of the assay is 0.2 ng/mL.

Total sHsp70 levels in serum samples of humans were measured using a modified Hsp70 immunoassay (Duoset, DYC1663, R&D Systems, Minneapolis, Minn., USA). The ELISA is designed to detect human Hsp70 in buffer.

### HSP-90

HSP90 ELISA (SUNRED Biotechnology Company, Shangai, People Republic of China) uses a double-antibody sandwich enzyme-linked immunosorbent assay to determine the level of human HSP90 in samples.

Serum HSP90a levels were determined in duplicate by a solid-phase ELISA technique (Assay Designs, Stressgen, Mich., USA).

Sandwich ELISA assays were performed in 96-well plates, using 100 μL peripheral blood plasma per well. When required, plasma was diluted in blocking buffer, in which case the concentration read from the standard curve was multiplied by the dilution factor. The following ELISA assays were used according to manufacturer recommendations: Hsp90a (human) ELISA Kit (Enzo Life Sciences, Farmingdale, N.Y.), Human beta 2-Microglobulin Quantikine IVD ELISA Kit (R&D Systems Inc., Minneapolis, Minn.) and Human IGFBP-2 DuoSet (R&D Systems In).

Lipids: 1,2-dioleoyl-sn-glycero-3-phosphocholine (DOPC) and cholesterol were purchased from Avanti Polar Lipids, Inc. (Alabaster, USA) and used as received. All other solvents (AR grade) were used without purification, and all reactions were carried out under nitrogen.

### Tethered Phospholipid Bilayer Membrane Formation

Gold substrates for electrochemical impedance spectroscopy (EIS) measurements were prepared on 25×75 mm glass slides (ThermoFischer Scientific, UK). Gold substrates for SPR were prepared on BK7 glass slides (25 mm diameter, 1 mm thickness) obtained from AutoLab (Methorm, The Netherlands). Gold layers were deposited by the magnetron sputtering using PVD75 (Kurt J. Lesker Co., U.S.) system. The details on the synthesis and properties of the self-assembled monolayers used in this work were described earlier.

Tethered lipid membranes (tBLM) were prepared by multilamellar vesicle fusion method described in previous papers. Vesicle solution was prepared from 1,2-dioleoyl-sn-glycero-3-phosphocholine (DOPC) and cholesterol (Avanti Polar Lipids, USA) in molar % ratio 6:4 and 7.5:2.5 in phosphate buffer solution (PBS) containing 0.1 M NaCl (Roth, Denmark), 0.01 M NaH2PO4 (Roth, Denmark), pH 4.5.

### Electrochemical Impedance Spectroscopy

EIS measurements were registered using Zennium electrochemical workstation (Zahner GmbH, Germany) between 0.1 Hz and 100 kHz, with 10 logarithmically distributed measurement points per decade. Data were fitted using ZView software (Scribner Associates, Southern Pines, N.C.). All measurements were carried out in sodium phosphate buffer solution (PBS) 0.1 M NaCl, 0.01 M NaH2PO4 pH 7.2. A three-electrode cell configuration was used. Reference electrode was Ag/AgCl, NaCl (sat.) electrode. Platinum wire (Aldrich, 99.99% purity) was used as auxiliary electrode. Working electrode was Au coated glass slide. In this work 12 vial electrochemical cell was used. Surface area of the working electrode exposed to the solution in the cell was 0.25 cm². All measurements were performed at room temperature (21±2° C.).

### Surface Plasmon Resonance (SPR)

The SPR measurements were conducted on an Autolab Twingle system (Eco Chemie B.V., The Netherlands) equipped with a flow-through cell (volume—175 μL). The unit performs SPR spectra recording at a fixed wavelength of 670 nm. It automatically follows with a millidegree resolution the position of an incidence angle (ranging from 62 to 78°), at which minimum of the reflection due to an excitation of the surface plasmon resonance is observed. Model F34 refrigerating/heating circulator (Julabo, DEU) was used to stabilize temperature at 21±0.1° C.

Before each experiment, the baseline in buffer solution was recorded. All measurements were carried out at stopped-flow conditions. According to manufacturer's manual minimum shift by 122 millidegrees (m°) corresponds to a surface coverage of 100 ng/cm2 of an adsorbed protein. So, in this work, we used the relation 1.22 m°=1 ng/cm2 to recalculate the amount of adsorbed protein per surface area from the SPR data.

### Results

Interaction of HSPs with Phospholipid Model Membranes by EIS and SPR.

The rise of the HSPs in the serum and urea is an important step in the pathogenesis of Acute pancreatitis. However, the mechanism by which HSP70 and HSP90 acts during AP is largely unknown. Data in the previous section suggests strong interaction of HSP70 and HSP90 preparations with phospholipid membranes.

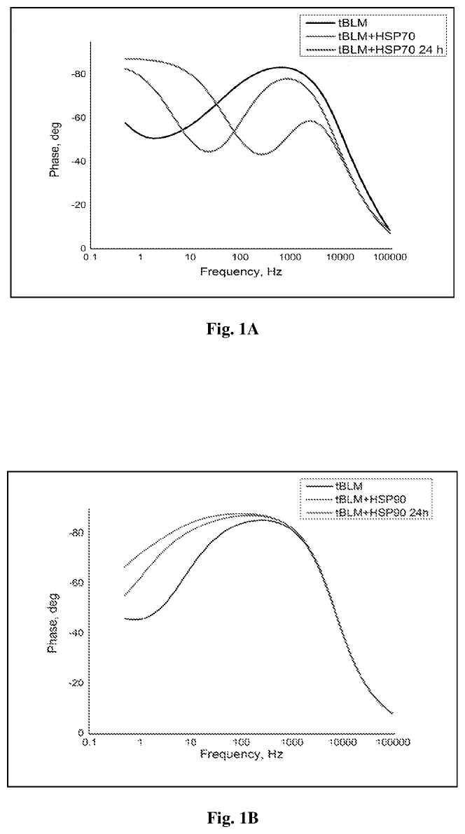

The effect of reconstitution of HSP70 and HSP90 into the phospholipid membrane is shown in FIG. 1, protein action is readily seen form the changes of EIS plots. In particular, upon incubation of tBLMs with HSP70 (FIG. 1A). In addition, the phase minimum appears, and it moves towards higher frequencies as the incubation time of HSP70 increases (FIG. 1A). The conductance (Y) of the tBLM, which is the HSP70 induced conductance at the phase minimum (estimated as Y=1/Zphase min). Phase shift after interaction with HSP70 shows the damage of the membrane and alters the ion movement through the membrane (FIG. 1A).

Interaction of Hsp90 with phospholipid model membranes is showed in FIG. 1B. The opposite result comparing with interaction of HSP70 was observed. The phase minimum appears, and it moves towards lower frequencies as the incubation time of HSP90 increases (FIG. 1B). Such phase shift after interaction with HSP90 shows that protein is attaching the membrane and isolating any ion movement through the membrane (FIG. 1B).

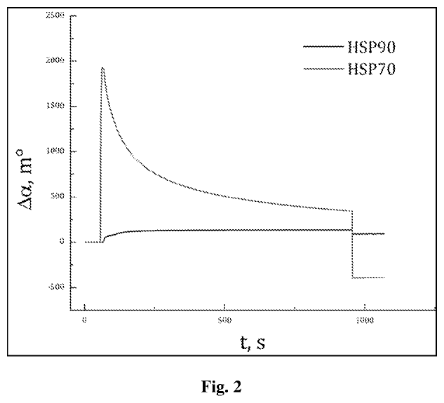

FIG. 2 displays representative SPR traces obtained on tBLM exposed to the solutions of HSP70 (red curve) and HSP90 (black curve) monomers. In HSP70 case, SPR signal after washing (at 900th second) dropped below the measurement start. These changes indicate a decrease in the lipids on the surface of the sensor. This is an unusually unexpected result, because it was not known that proteins would disrupt the lipid membrane and this behaviour can be associated only with the specific protein properties. While another HSP90 protein causes an increase in the SPR signal. These changes are related to the adsorptions on lipids membrane.

Interaction of Hsp90 with phospholipid model membranes is showed in FIG. 1B. The opposite result comparing with interaction of HSP70 was observed. The phase minimum appears, and it moves towards lower frequencies as the incubation time of HSP90 increases (FIG. 1B). Such phase shift after interaction with HSP90 shows, that protein is attaching the membrane and isolating any ion movement through the membrane (FIG. 1B).

### REFERENCES

1. Bhagat, Lakshmi, Vijay P. Singh, Rajinder K. Dawra, and Ashok K. Saluja. 2008. ‘Sodium Arsenite Induces Heat Shock Protein 70 Expression and Protects against Secretagogue-Induced Trypsinogen and NF-KappaB Activation.’ Journal of Cellular Physiology 215 (1): 37-46. https://doi.org/10.1002/jcp.21286.
2. Bhagat, Lakshmi, Vijay P. Singh, Albert M. Song, Gijs J. D. van Acker, Sudhir Agrawal, Michael L. Steer, and Ashok K. Saluja. 2002. ‘Thermal Stress-Induced HSP70 Mediates Protection against Intrapancreatic Trypsinogen Activation and Acute Pancreatitis in Rats.’ Gastroenterology 122 (1): 156-65.
3. Bhatia, Madhav, Fei Ling Wong, Yang Cao, Hon Yen Lau, Jiali Huang, Padmam Puneet, and Lakshmi Chevali. 2005. ‘Pathophysiology of Acute Pancreatitis.’ Pancreatology: Official Journal of the International Association of Pancreatology (IAP) . . . [et Al.] 5 (2-3): 132-44. https://doi.org/10.1159/000085265.
4. Budvytyte, Rima, Gintaras Valincius, Gediminas Niaura, Vladislava Voiciuk, Mindaugas Mickevicius, Hilary Chapman, Haw-Zan Goh, et al. 2013. ‘Structure and Properties of Tethered Bilayer Lipid Membranes with Unsaturated Anchor Molecules’. Langmuir: The ACS Journal of Surfaces and Colloids 29 (27): 8645-8656. https://doi.org/10.1021/1a401132c.
5. Cikotiene, Inga, Egidijus Kazlauskas, Jurgita Matuliene, Vilma Michailoviene, Jolanta Torresan, Jelena Jachno, and Daumantas Matulis. 2009. ‘ChemInform Abstract: 5-Aryl-4-(5-Substituted-2,4-Dihydroxyphenyl)-1,2,3-Thiadiazoles as Inhibitors of Hsp90 Chaperone.’ ChemInform 40 (28). https://doi.org/10.1002/chin.200928122.
6. Dudeja, V., S. M. Vickers, and A. K. Saluja. 2009. ‘The Role of Heat Shock Proteins in Gastrointestinal Diseases.’ Gut 58 (7): 1000-1009. https://doi.org/10.1136/gut.2007.140194.
7. Dymock, Brian W, Martin J Drysdale, Edward McDonald, and Paul Workman. 2004. ‘Inhibitors of HSP90 and Other Chaperones for the Treatment of Cancer’. Expert Opinion on Therapeutic Patents 14 (6): 837-47. https://doi.org/10.1517/13543776.14.6.837.
8. Ethridge, R. T., R. A. Ehlers, M. R. Hellmich, S. Rajaraman, and B. M. Evers. 2000. ‘Acute Pancreatitis Results in Induction of Heat Shock Proteins 70 and 27 and Heat Shock Factor-1.’ Pancreas 21 (3): 248-56.
9. Fagenholz, Peter J., Carlos Fernández-Del Castillo, N. Stuart Harris, Andrea J. Pelletier, and Carlos A. Camargo. 2007. ‘Increasing United States Hospital Admissions for Acute Pancreatitis, 1988-2003’. Annals of Epidemiology 17 (7): 491.e1-491.e8. https://doi.org/https://doi.org/10.1016/j.annepidem.2007.02.002.
10. Fagenholz, Peter J., Carlos Ferna\acute{ }ndez-del Castillo, N. Stuart Harris, Andrea J. Pelletier, and Carlos A. Jr Camargo. 2007. ‘Direct Medical Costs of Acute Pancreatitis Hospitalizations in the United States’. Pancreas 35 (4). https://journals.lww.com/pancreasjournal/Fulltext/2007/11000/Direct_Medical_Costs_of_Acute_Pancreatitis.2.aspx.
11. Feng, Jia Yan, and Yong Yu Li. 2010. ‘Alteration and Role of Heat Shock Proteins in Acute Pancreatitis’. Journal of Digestive Diseases 11 (5): 277-83. https://doi.org/10.1111/j.1751-2980.2010.00450.x.
12. Fu, K., M. P. Jr Sarras, R. C. De Lisle, and G. K. Andrews. 1997. ‘Expression of Oxidative Stress-Responsive Genes and Cytokine Genes during Caerulein-Induced Acute Pancreatitis.’ The American Journal of Physiology 273 (3 Pt 1): G696-705. https://doi.org/10.1152/ajpgi.1997.273.3.G696.
13. Giri, Bhuwan, Vrishketan Sethi, Shrey Modi, Bharti Garg, Sulagna Banerjee, Ashok Saluja, and Vikas Dudeja. 2017. ‘“Heat Shock Protein 70 in Pancreatic Diseases: Friend or Foe”.’ Journal of Surgical Oncology 116 (1): 114-22. https://doi.org/10.1002/jso.24653.
14. Gress, T. M., F. Muller-Pillasch, C. Weber, M. M. Lerch, H. Friess, M. Buchler, H. G. Beger, and G. Adler. 1994. ‘Differential Expression of Heat Shock Proteins in Pancreatic Carcinoma.’ Cancer Research 54 (2): 547-51.
15. Iovanna, Juan, and U Ismailov. 2009. Pancreatology: From Bench to Bedside. https://doi.org/10.1007/978-3-642-00152-9.
16. Lee, Hong Sik, Lakshmi Bhagat, Jean-Louis Frossard, Antti Hietaranta, Vijay P. Singh, Michael L. Steer, and Ashok K. Saluja. 2000. ‘Water Immersion Stress Induces Heat Shock Protein 60 Expression and Protects against Pancreatitis in Rats’. Gastroenterology 119 (1): 220-29. https://doi.org/https://doi.org/10.1053/gast.2000.8551.
17. Lerch, Markus M., and Fred S. Gorelick. 2013. ‘Models of Acute and Chronic Pancreatitis.’ Gastroenterology 144 (6): 1180-93. https://doi.org/10.1053/j.gastro.2012.12.043.
18. McGillivray, Duncan J., Gintaras Valincius, David J. Vanderah, Wilma Febo-Ayala, John T. Woodward, Frank Heinrich, John J. Kasianowicz, and Mathias Lösche. 2007. ‘Molecular-Scale Structural and Functional Characterization of Sparsely Tethered Bilayer Lipid Membranes’. Biointerphases 2 (1): 21-33. https://doi.org/10.1116/1.2709308.
19. Niederau, C., L. D. Ferrell, and J. H. Grendell. 1985. ‘Caerulein-Induced Acute Necrotizing Pancreatitis in Mice: Protective Effects of Proglumide, Benzotript, and Secretin.’ Gastroenterology 88 (5 Pt 1): 1192-1204.
20. Ogata, M., Z. Naito, S. Tanaka, Y. Moriyama, and G. Asano. 2000. ‘Overexpression and Localization of Heat Shock Proteins MRNA in Pancreatic Carcinoma.’ Journal of Nippon Medical School=Nippon Ika Daigaku Zasshi 67 (3): 177-85.
21. Peery, Anne F., Evan S. Dellon, Jennifer Lund, Seth D. Crockett, Christopher E. McGowan, William J. Bulsiewicz, Lisa M. Gangarosa, et al. 2012. ‘Burden of Gastrointestinal Disease in the United States: 2012 Update.’ Gastroenterology 143 (5): 1179-1187.e3. https://doi.org/10.1053/j.gastro.2012.08.002.
22. Ragaliauskas, Tadas, Mindaugas Mickevicius, Bozena Rakovska, Tadas Penkauskas, David J. Vanderah, Frank Heinrich, and Gintaras Valincius. 2017. ‘Fast Formation of Low-Defect-Density Tethered Bilayers by Fusion of Multilamellar Vesicles’. Biochimica et Biophysica Acta (BBA)—Biomembranes 1859 (5): 669-78. https://doi.org/https://doi.org/10.1016/j.bbamem.2017.01.015.
23. Rakonczay, Zoltan Jr, Bela Ivanyi, Ilona Varga, Imre Boros, Andrea Jednakovits, Ilona Nemeth, Janos Lonovics, and Tamas Takacs. 2002. ‘Nontoxic Heat Shock Protein Coinducer BRX-220 Protects against Acute Pancreatitis in Rats.’ Free Radical Biology & Medicine 32 (12): 1283-92.
24. Rakonczay, Zoltan Jr, Yvette Mandi, Jozsef Kaszaki, Bela Ivanyi, Imre Boros, Janos Lonovics, and Tamas Takacs. 2002. ‘Induction of HSP72 by Sodium Arsenite Fails to Protect against Cholecystokinin-Octapeptide-Induced Acute Pancreatitis in Rats.’ Digestive Diseases and Sciences 47 (7): 1594-1603.
25. Rakonczay, Zoltán, Tamás Takács, Imre Boros, and János Lonovics. 2003. ‘Heat Shock Proteins and the Pancreas’. Journal of Cellular Physiology 195 (3): 383-91. https://doi.org/10.1002/jcp.10268.
26. Sah, Raghuwansh P., and Ashok Saluja. 2011. ‘Molecular Mechanisms of Pancreatic Injury.’ Current Opinion in Gastroenterology 27 (5): 444-51. https://doi.org/10.1097/MOG.0b013e328349e346.
27. Sato, H., R. C. Siow, S. Bartlett, S. Taketani, T. Ishii, S. Bannai, and G. E. Mann. 1997. ‘Expression of Stress Proteins Heme Oxygenase-1 and -2 in Acute Pancreatitis and Pancreatic Islet BetaTC3 and Acinar AR42J Cells.’ FEBS Letters 405 (2): 219-23.
28. Strowski, M. Z., G. Sparmann, H. Weber, F. Fiedler, H. Printz, L. Jonas, B. Goke, and A. C. Wagner. 1997. ‘Caerulein Pancreatitis Increases MRNA but Reduces Protein Levels of Rat Pancreatic Heat Shock Proteins.’ The American Journal of Physiology 273 (4 Pt 1): G937-945.
29. Weber, C. K., T. Gress, F. Muller-Pillasch, M. M. Lerch, H. Weidenbach, and G. Adler. 1995. ‘Supramaximal Secretagogue Stimulation Enhances Heat Shock Protein Expression in the Rat Pancreas.’ Pancreas 10 (4): 360-67.
30. Whitcomb, D. C. 2004. ‘Value of Genetic Testing in the Management of Pancreatitis.’ Gut 53 (11): 1710-17. https://doi.org/10.1136/gut.2003.015511.
31. Workman, Paul. 2004. ‘Altered States: Selectively Drugging the Hsp90 Cancer Chaperone.’ Trends in Molecular Medicine 10 (2): 47-51.
32. Yadav, Dhiraj, and Albert B. Lowenfels. 2006. ‘Trends in the Epidemiology of the First Attack of Acute Pancreatitis: A Systematic Review.’ Pancreas 33 (4): 323-30. https://doi.org/10.1097/01.mpa.0000236733.31617.52.

1. A method for determining acute pancreatitis disease initiation, wherein the method comprises the steps of measuring the levels of heat shock proteins HSP-70, 90, 60, 27 in human urine or other body fluids samples of patients admitted to emergency department within 72 hours since the onset of the symptoms of acute pancreatitis, analysing the data according to protocol, combining the data with routine chemical tests to provide a clinical diagnosis.

2. A method for determining lifetime risk for acute pancreatitis, wherein genetic testing of HSP-70, 90, 60, and 27 is performed for a subject in risk of AP.

3. A method according to claim 1 additionally involving a non-invasive radiological test to determine the need for surgical intervention.
